SEQUENCE LISTING (3 sequences)

SEQ ID NO: 1 - protein, 4 aa, Artificial
  Artificial Sequence - Synthetic
ECVG

SEQ ID NO: 2 - protein, 4 aa, Artificial
  Artificial Sequence - Synthetic
ECKG

SEQ ID NO: 3 - protein, 5 aa, Artificial
  Artificial Sequence - Synthetic
ECACG

(19) United States
(12) Patent Application Publication
(10) Pub. No.: US 2008/0200563 A1
(43) Pub. Date: Aug. 21, 2008

(54) Use of Compounds Containing Thiol Groups as Efflux Pump Inhibitors

(75) Inventor: Martin Hoffer, Vienna (AT)

Correspondence Address: LOWRIE, LANDO & ANASTASI, LLP, ONE MAIN STREET, SUITE 1100, CAMBRIDGE, MA, 02142, US

(73) Assignees: ThioMatrix Forschungs- und Beratungs- GmbH, Innsbruck (AT); MucoBiomer Biotechnologische Forschungs- und Entwicklungs- GmbH, Leobendorf (AT)

(21) Appl. No.: 11/632,868

(22) PCT Filed: Jul. 14, 2005
(86) PCT No.: PCT/EP2005/053395

§ 371 Date: Jan. 19, 2007

(30) Foreign Application Priority Data
Jul. 22, 2004 (AT) A 1250/2004

(51) Int. Cl. A61K 47/32; A61K 47/38; A61K 47/26; A61K 47/42

(52) U.S. Cl. 514/772.6; 514/781; 514/777; 514/773

(57) ABSTRACT

The non-invasive administration of many active ingredients fails with efflux pumps which sharply reduce the active ingredient absorption on mucous membranes. According to the invention, the active ingredient absorption on mucous membranes can be drastically improved by using dosages containing glutathione and/or compounds comprising numerous thiol groups, in addition to the active ingredient(s). Forms of administration such as matrix tablets, capsules, eye drops, or microparticles, containing the cited combination of active ingredients and auxiliary substances, can be used.

Non-invasive dosage forms are in comparison to parenteral dosage forms much more convenient for patients. By the administration of tablets or nasal sprays, for instance, pain and risks being associated with e.g. injections and infusions can be avoided. Accordingly higher is the compliance of non-invasive dosage forms. Many classes of drugs such as chemotherapeutics used for cancer treatment, however, can only be administered parenterally, as their absorption via mucosal membranes is strongly reduced by various efflux pumps such as for instance P-glycoprotein [Hunter, J. and Hirst, B. H. (1997) Intestinal secretion of drugs. The role of P-glycoprotein and related drug efflux Systems in limiting oral drug absorption. Adv. Drug Deliv. Rev., 25, 129-157]. Moreover, the bioavailability of various further drugs from the class of antiarrhythmics, antibiotics, antimycotics, anticoagulants, antimalarial drugs, calcium channel blockers and immunosuppressive drugs is in case of administration via mucosal membranes also strongly reduced by efflux pumps. The corneal absorption of erythromycin, for instance, is strongly reduced by P-glycoprotein [Dey S, Gunda S, Mitra A K. Pharmacokinetics of Erythromycin in Rabbit Corneas Following Single-Dos[deg.]e Infusion. Role of P-Glycoprotein as a Barrier to in vivo Ocular Drug Absorption. J Pharmacol Exp Ther. 2004, in press; Dey S, Patel J, Anand B S, Jain-Vakkalagadda B, Kaliki P, Pal D, Ganapathy V, Mitra A K., Molecular Evidence and Functional Expression of P-Glycoprotein (MDR1) in Human and Rabbit Cornea and Corneal Epithelial Cell Lines, Invest Ophthalmol Vis Sei. 2003 JuI; 44 (7) 2909-18]. The development of dosage forms to overcome these efflux pumps is therefore subject of intensive research activities since many years.

Due to the administration of paclitaxel via a self-microemulsifying dosage form, for instance, its oral bioavailability was improved from 28.6% up to 52.7% [Yang, S., Gursoy R. N., Lambert G. and Benita S., Enhanced oral absorption of padlitaxel in a novel self-microemulsifying drug delivery System with or without conemitant use of P-glycoprotein inhibitors. Pharm. Res. 21 (2004) 261-270]. In another study, for instance, the oral bioavailability of cyclosporin was significantly improved by the use of a nanoparticulate dosage form [Beckerman T., Golenser J. and Domb A., Cyclosporin nanoparticulate lipospheres for oral administration. J. Pharm. Sei. 93 (2004) 1264-1270]. In further attempts to solve these problems various inhibitors of gastrointestinal efflux pumps could be identified [e.g. Dintaman, J. M. and Silverman, J. A. (1999). Inhibition of P-glycoprotein by D-alpha-tocopheryl polyethylene glycol 1000 succinate (TPGS). Pharm. Res. 16, 1550-1556; Senior, A. E., Gros, P., and Urbatsch, I. L. (1998). Residues in P-glycoprotein catalytic sites that react with the inhibitor 7-chloro-4-nitrobenzo-2-oxa1,3-diazole. Arch. Biochem. Biophys., 357, 121-125].

The efficacy of already developed dosage forms, however, is in many cases only very poor, so that for instance most cytostatic drugs still have to be administered parenterally. Therefore, it is the object of the present invention to provide pharmaceutical dosage forms for drugs which are poorly absorbed by mucosal membranes because of efflux pumps, wherein these new dosage forms counteract the reduced absorption.

The present invention relates to the field of pharmaceutical technology. The invention is based on completely novel dosage forms comprising at least one excipient in addition to the drug, the excipient having numerous thiol groups within its chemical structure. Permeation studies showed completely unexpectedly a very efficient inhibition of efflux pumps on mucosal membranes by adding such polythiols. This effect can even be further improved by the addition of glutathione. Moreover glutathione shows per se a significant inhibitory effect on efflux pumps on mucosal membranes. In order to avoid too rapid an absorption or dilution of polythiol-compounds for instance in the gastrointestinal tract, these compounds should preferably have a molecular weight of more than 2 kDa and typically more than 100 kDa. Glutathione is generally hardly resorbed from mucosal membranes [Langoth N., Development of buccal drug delivery Systems for peptide drugs, Dissertation, University of Vienna, 2003]. As dosage forms matrix-tablets, eye drops and microparticles have been developed which contain polythiol-compound(s) and/or glutathione in addition to the drug. Only by the combined use of the drug and the excipients in an appropriate dosage form the intended effect can be achieved. Apart from an oral administration also an ocular, nasal, pulmonary and rectal administration of these novel dosage forms is of commercial interest. Pharmaceutical compositions comprising glutathione as stabilizing agent are described e.g. in WO 95/19177 A, JP 1/203336 A, JP 1/022817 A and JP 57/058616 A.

Accordingly, the present invention also relates to pharmaceutical dosage forms for an improved drug absorption, said dosage forms containing: one or more drugs of limited uptake via mucosal membranes because of efflux pumps, glutathione or a derivative thereof, and/or at least one compound used as an excipient, which consists of not more than 10 different subunits and which bears at least 10 covalently bound thiol groups in its chemical structure.

According to a preferred embodiment, the pharmaceutical dosage forms according to this invention contain apart from glutathione or a derivative thereof and/or apart from the polythiol-compound no additional thiol compound.

Especially preferred according to the invention is thereby a combination of glutathione or a derivative thereof with a polythiol-compound, as this combination shows a particularly high efflux pump inhibitory effect.

The present invention is useful for all drugs, the absorption of which is inhibited or at least reduced by efflux pumps. The present invention is in particular useful for all drugs being substrates of efflux pumps and for which a parenteral administration is disadvantageous or inconvenient for patients. According to the present invention, preferred dosage forms are therefore those containing one or more drugs selected from the group of chemotherapeutics, antiarrhythmics, antibiotics, antiinflammatory drugs, local anesthetic drugs, hormones, antimycotics, anticoagulants, antimalarial drugs, calcium channel blockers, immunosuppressive drugs and fluorescence markers and in particular those containing taxol, cyclosporin, saquinavir or ritonavir. Pharmaceutical dosage forms containing as polythiol compound(s) thiol group containing derivatives of carbomer, poly(meth)arylic acid, poly(D-glucosamine), cellulose, polylysine or polyarginine, which have more than 10 and typically more than 100 thiol groups in their chemical structure, are preferred embodiments of the present invention, as they show a superior inhibitory effect on efflux pumps. Consequently, drugs being administered with such dosage forms are absorbed particularly well and in high quantities. Preferably, the polythiol-compound(s) used have a molecular weight of more than 2 kDa and typically more than 100 kDa.

According to the present invention, preferred dosage forms comprise nanoparticles, microparticles, matrix-tablets, emulsions, solutions, suspensions, eye drops and capsules as well as pharmaceutical dosage forms for oral, nasal, pulmonary, vaginal, buccal, rectal and ocular application.

According to a certain aspect, the present invention relates to the use of pharmaceutically acceptable thiol groups containing compounds for the manufacture of medicaments for inhibiting efflux pumps, the medicaments containing drugs, the mucosal uptake of which is inhibited by efflux pumps in the absence of thiol groups containing compounds. Hence, formulations according to this invention can be regarded as combination medicaments containing at least two active ingredients: a first active ingredient comprising a certain drug for the prevention or treatment of a certain disease or dysfunction of an individual; and a second active ingredient being responsible for the inhibition of efflux pumps which counteract the uptake of said first active ingredient, in a way that the first active ingredient can be efficiently taken up from the body of the individual. Preferred efflux pumps, which can be inhibited according to the present invention, are those being described as pharmaceutically relevant. Thereby, the inhibition of P-glycoprotein, ABCG2, ABCC1 and ABCC2 and among them especially P-glycoprotein is particularly preferred. According to the present invention, either the effective dose of the first active ingredient can be increased in the body or the amount of the first active ingredient can be reduced in certain medicaments without leading to a reduced efficacy.

According to the present invention, the first active ingredient is a drug (or a combination of drugs), the uptake of which is usually (i.e. in the absence of the thiol groups containing compounds to be used according to the present invention) reduced by efflux pumps (i.e. being instantly secreted instead of being supplied to the individuum via mucosal membranes or other biological barriers bearing efflux pumps (e.g. blood-brain barrier, cancer cells, etc.) (systemic or local)). This efflux of the drug is inhibited or reduced by the second active ingredient (one or more thiol group containing compounds) guaranteeing an improved absorption of the drug (in comparison to the absorption without the thiol group containing compound). According to the present invention, the first active ingredient can be all drugs or drug combinations of limited absorption from mucosal membranes due to the activity of efflux pumps. Such drugs show, for instance, an efflux ratio (secretory apparent permeability coefficient/absorptive apparent permeability coefficient) of >1.5, preferred >2.0, and in particular >2.5 (e.g. in a test system as described in example 1 at 37°).

Very appropriate thiol group containing compounds according to the present invention have a molecular weight of at least 250 g/mol. Compounds exhibiting a lower molecular weight are less advantageous with respect to their thiol group dependent efflux pump inhibitory properties.

Preferred thiol group containing compounds according to the present invention display on the one hand at least one thiol group per 1000 g/mol molecular weight and in particular one thiol group per 500 g/mol molecular weight. On the other hand, the thiol group containing compound should bear at least 10 thiol groups per molecule, especially if greater molecules are utilized.

Preferred is the use of compounds being composed of one or more monomer units, wherein at least one monomer unit can be thiolated. Thiol group containing compounds according to the present invention, however, are preferably composed of not more than 10 different subunits. Typically, they shall be composed of one, two or three different monomer units. Especially useful are compounds being in their applicability as pharmaceutical ingredients already well-known and documented, or physiologically acceptable thiolated derivatives thereof, i.e. those compounds which may be prepared by pharmaceutical formulation agents by introducing thiol groups.

According to the present invention, preferred thiol group containing compounds are selected from thiolated carbomer, thiolated poly(meth)acrylic acid, thiolated cellulose, thiolated polyglucosamines, thiolated polylysines, thiolated polyarginines or glutathione or glutathione derivatives, typically those glutathione derivatives having the —SH and —COOH group, in particular having both —COOH groups (e.g. compounds such as Glu-Cys-Val-Gly, Glu-Cys-Lys-Gly, Glu-Cys-Ala-Cys-Gly).

The described combination medicament can usually also be administered as a mixture of the drug and the thiol group containing compound. Both compounds have thereby to be used in a sufficient high dose in order to guarantee on the one hand the intended therapeutic effect and on the other hand a sufficient inhibition of efflux pumps. The separate administration where the drug and the efflux pump inhibiting compound are administered separately, or, for instance, as kit, however, is also possible.

The invention is illustrated in more detail by the following examples and figures, but not limited to them.

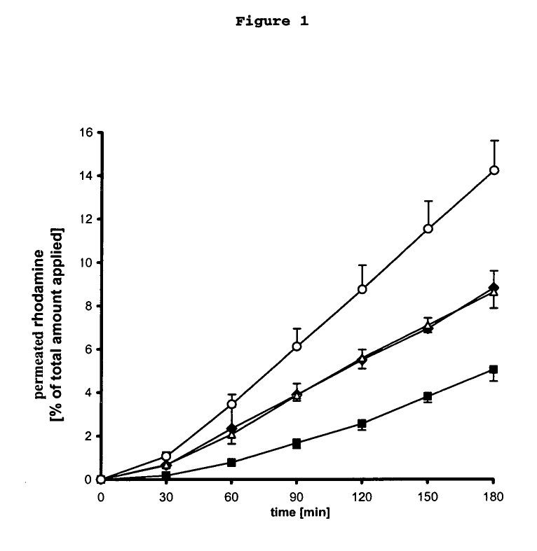

FIG. 1 shows the results of permeation studies with rhodamine 123; Transport across the mucosa in the absorptive (apical to basolateral; black symbols) and secretory direction (basolateral to apical; white symbols) in the absence (▪, ◯) or presence (♦, Δ) of glutathione at a concentration of 0.5% (m/v).

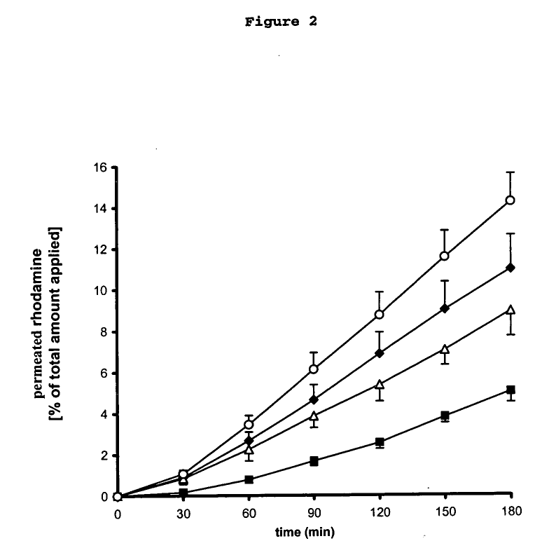

FIG. 2 shows the results of permeation studies with rhodamine 123; Transport across the mucosa in the absorptive (apical to basolateral; black symbols) and secretory direction (basolateral to apical; white symbols) in the absence (▪, ◯) or presence (♦, Δ) of poly(D-glucosamine)-cysteine at a concentration of 0.5% (m/v).

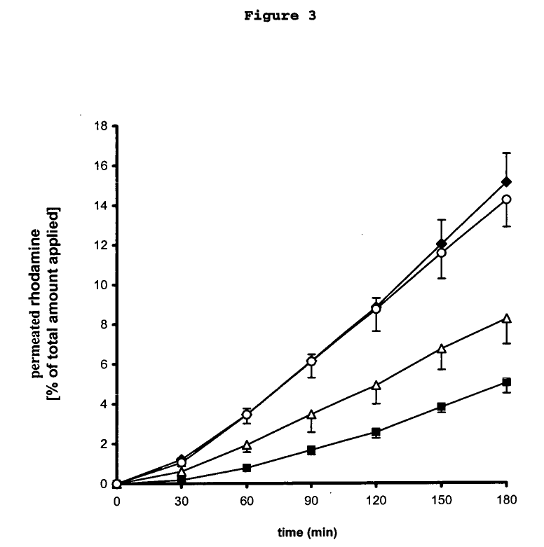

FIG. 3 shows the results of permeation studies with rhodamine 123; Transport across the mucosa in the absorptive (apical to basolateral; black symbols) and secretory direction (basolateral to apical; white symbols) in the absence (▪, ◯) or presence (♦, Δ) of the combination of poly(D-glucosamine)-cysteine (0.5%; m/v) and glutathione (0.5%; m/v).

### EXAMPLES

### Example 1

### Inhibition of Efflux Pumps on the Mucosa by Glutathione

The small intestine of guinea pigs was immediately removed after sacrificing animals, cut lengthwise and rinsed with sterile 0.9% sodium chloride solution. Thereafter it was mounted in Ussing chambers. The incubation medium was a 50 mM Bis-Tris (bis[2-hydroxyethyl]imino-tris[hydroxymethyl]methane) pH 6.0 buffer containing 250 mM NaCl, 2.6 mM MgSO₄, 10 mM KCl, 40 mM glucose and 50 mM NaHCO₃. The Ussing chambers were gassed with a mixture of 95% O₂ and 5% CO₂ and maintained at a temperature of 37° C. After an equilibration period of 30 minutes rhodamine 123 being reported in the literature as a substrate for the efflux pump P-glycoprotein [e.g. Tang F, Ouyang H, Yang J Z, Borchardt R T., Bidirectional transport of rhodamine 123 and Hoechst 33342, fluorescence probes of the binding sites on P-glycoprotein, across MDCKMDR1 cell monolayers. J Pharm Sei. 2004 May; 93 (5) 1185-94] was added to the apical compartment in a final concentration of 0.001% (m/v). At predetermined time points samples were withdrawn from the acceptor chamber—facing to the basolateral site of the mucosa and replaced by fresh incubation medium. The concentration of permeated rhodamine 123 was determined by using a fluorimeter. In addition all permeation studies described above were also performed with the mucosa being mounted in the opposite direction, so that the donor compartment is facing the basolateral site of the mucosa. In parallel, the same experiments were performed with the only difference that the donor- and acceptor compartment contained additionally glutathione in a concentration of 0.5%.

The results of these permeation studies are shown in FIG. 1. In this Figure, the transport of rhodamine 123 across the mucosa in absorptive direction (apical to basolateral; black symbols) and in the secretory direction (basolateral to apical; white symbols) in the absence (▪, ◯) or presence (♦, Δ) of glutathione in a concentration of 0.5% (m/v) is illustrated. Indicated values are expressed as percentage of the total dose of rhodamine (0.001%; m/v) applied, which was able to permeate the mucosa. Indicated values are mean values±SD of at least three experiments. Within the study a statistically significant (p<0.05) efflux pump inhibitory effect of glutathione was shown, as the permeation of rhodamine in the absorptive direction in the presence of glutathione was significantly improved, whereas it was significantly reduced in the secretory direction in the presence of glutathione.

Performing experiments as described above at 4° C. showed a dramatically lower effect of glutathione indicating the inhibition of efflux pumps.

### Example 2

### Inhibition of Efflux Pumps on the Mucosa by a Polythiol Compound

Permeation studies in the presence of a polythiol compound were performed as described in example 1. The efflux pump inhibitor glutathione, however, was substituted by the polythiol compound poly(D-glucosamine)-cysteine (MucoBiomer GmbH, Leobendorf, A) in a final concentration of 0.5% (m/v).

In FIG. 2, the transport of rhodamine 123 across the mucosa in absorptive direction (apical to basolateral; black symbols) and in the secretory direction (basolateral to apical; white symbols) in the absence (▪, ◯) and presence (♦, Δ) of poly(D-glucosamine)-cysteine in a concentration of 0.5% (m/v) is illustrated. Indicated values are expressed as percentage of the total dose of rhodamine (0.001%; m/v) applied, which was able to permeate the mucosa. Indicated values are mean values±SD of at least three experiments. Within the study a statistically significant (p<0.05) efflux pump inhibitory effect of the polythiol compound was shown, as the permeation of rhodamine in the absorptive direction in the presence of the polythiol compound was significantly improved, whereas it was significantly reduced in the secretory direction in the presence of the polythiol compound.

Performing experiments as described above at 4° C. showed a dramatically lower effect of the polythiol compound indicating the inhibition of efflux pumps.

### Example 3

Inhibition of Efflux Pumps on the Mucosa by the Combined Use of Glutathione with a Polythiol Compound

Permeation studies in the presence of glutathione and of a polythiol compound were performed as described in example 1. Apart from the efflux pump inhibitor glutathione, however, also the polythiol compound poly(D-glucosamine)-cysteine (MucoBiomer GmbH, Leobendorf, A) was added in a final concentration of 0.5% (m/v).

In FIG. 3, the transport of rhodamine 123 across the mucosa in absorptive direction (apical to basolateral; black symbols) and in the secretory direction (basolateral to apical; white symbols) in the absence (▪, ◯) and presence (♦, Δ) of glutathione and of poly(D-glucosamine)-cysteine both applied in a concentration of 0.5% (m/v) is illustrated. Indicated values are expressed as percentage of rhodamine (0.001%; m/v) applied, which was able to permeate the mucosa. Indicated values are mean values±SD of at least three experiments. Within the study a statistically significant (p<0.05) efflux pump inhibitory effect of the combination of glutathione and a polythiol compound was shown, as the permeation of rhodamine in the absorptive direction in the presence of the combination of glutathione and the polythiol compound was significantly improved, whereas it was significantly reduced in the secretory direction in the presence of the combination of glutathione and of poly(D-glucosamine)-cysteine.

Performing experiments as described above at 4° C. showed a dramatically lower effect of this combination indicating the inhibition of efflux pumps in the presence of this combination.

### Example 4

Comparison with Well-known Efflux Pump Inhibitors

In order to compare the inhibitory effect of glutathione and/or polythiol-compounds with well-known inhibitors on efflux pumps of the gastrointestinal mucosa, they were tested in parallel. Studies were performed under conditions as described in example 1. The adjusted temperature, concentration of each tested compound and the resulting effect on the mucosa are listed in the table shown below. The table shows a comparison of the absorptive and secretory apparent permeability coefficient (Papp) of rhodamine 123 and the resulting efflux ratio in presence and absence of the listed compounds. Indicated values are mean values of 3 experiments each.

| | | | Efflux ratio |
|---|---|---|---|
| | Pₐₚₚ (cm/s) × 10⁻⁶ | | (secretory |
| Test | Transport direction | | Pₐₚₚ/absorptive |
| conditions | absorptive | secretory | Pₐₚₚ) |
| Buffer (37° C.) | 7.31 ± 0.77 | 20.6 ± 1.98 | 2.8 |
| Buffer (4° C.) | 1.35 ± 0.17 | 1.32 ± 0.13 | 1.0 |
| Terfenadine | 12.2 ± 0.08 | 14.0 ± 1.94 | 1.1 |
| (50 μM; 37° C.) | | | |
| Verapamil | 12.5 ± 2.29 | 12.5 ± 2.03 | 1.0 |
| (100 μM; | | | |
| 37° C.)) | | | |
| Glutathione | 12.8 ± 1.13 | 12.5 ± 1.09 | 1.0 |
| (0.5%; 37° C.) | | | |
| Poly(D- | 15.9 ± 2.40 | 13.0 ± 1.77 | 0.8 |
| glucosamine) - | | | |
| cysteine | | | |
| (0.5%; 37° C.) | | | |
| Poly(D- | 21.9 ± 2.10 | 12.0 ± 1.84 | 0.5 |
| glucosamine) - | | | |
| cysteine/glutathione | | | |
| (each | | | |
| 0.5%; 37° C.) | | | |

### Example 5

### Preparation of Matrix-tablets

1 g of poly(D-glucosamine)-cysteine (MucoBiomer, Leobendorf, A) was homogenized with 0.5 g of glutathione (Sigma, Vienna, A) and 0.5 g of taxol (Sigma, Vienna, A) and directly compressed to tablets (diameter: 8 mm; depth: 4 mm). Dissolution studies performed with these tablets in a water/DMSO mixture used as release medium showed a controlled release of both the drug and glutathione from this drug delivery system.

### Example 6

### Preparation of Microparticles

0.3 g of cyclosporine and 1 g of the polythiol-compound carbomer-cysteine (MucoBiomer, Leobendorf, A) were swollen in demineralised water. Thereafter 100 ml of this solution were precipitated in one liter of acetone and the precipitate was washed with acetone several times. The precipitate was then lyophilized and grinded in a mortar. The resulting microparticles showed a size in the middle em-range and exhibited a favourable drug release.

### Example 7

### Preparation of Eye Drops

0.3 g of erythromycin, 0.1 g of poly(D-glucosamine)-cysteine (MucoBiomer GmbH, Leobendorf, A) and 0.5 g of glutathione (Sigma, Vienna, A) are dissolved in 100 ml of sterile water. Thereafter, isotony was adjusted by the addition of sodium chloride. The solution was filtered and filled in each 10 ml eye drop bottles.

### Example 8

### Preparation of Nasal Gels

1 g of the thiol compound carbomer-cysteine (MucoBiomer, Leobendorf, A) is swollen in demineralised and degassed water. Thereafter 0.01-0.5 g of Leu-enkephaline and 5 g of glutathione are added and the pH is adjusted to 5.4. The nasal gel is filled in tubes of 5 g each and inertly sealed.

1-14. (canceled)

15. An excipient selected to increase mucosal uptake of a drug, the excipient comprising an effective amount of a compound comprising at least one thiol group to inhibit efflux pumps.

16. The excipient of claim 15, in which the compound comprising at least one thiol group has a molecular weight of at least 250 g/mol.

17. The excipient of claim 15, in which the compound comprising at least one thiol group comprises at least 10 thiol groups.

18. The excipient of claim 15, in which the compound comprising at least one thiol group is selected from the group consisting of thiolated carbomer, thiolated poly(meth)acrylic acid, thiolated cellulose, thiolated polyglucosamines, thiolated polylysines, thiolated polyarginines, glutathione and combinations thereof.

19. The excipient of claim 15, in which the compound comprising at least one thiol group comprises at least one thiol group per 500 g/mol of molecular weight.

20. The excipient of claim 15, in which the compound comprising at least one thiol group is effective to reduce efflux ratio by at least 50% when being applied in a concentration of less than 100 μM.

21. The excipient of claim 15, in which the thiol containing compound is present an amount effective to inhibit one or more of P-glycoprotein, ABCG2, ABCC1 and ABCC2.

22. The excipient of claim 15, wherein said medicament preparation is a dosage form for oral, nasal, pulmonary, vaginal, buccal, rectal and ocular administration.

23. The excipient of claim 15, in which the thiol containing compound consists of glutathione.

24. A medicament preparation comprising a drug whose mucosal uptake is limited by efflux pumps and an effective amount of an excipient comprising a compound with at least one thiol group to inhibit the efflux pumps.

25. The medicament preparation of claim 24, in which said drug is one or more of a chemotherapeutic, an anti-arrhythmic, an antibiotic, an anti-inflammatory drug, a local anesthetic drug, a hormone, an antimycotic, an anticoagulant, an antimalarial drug, a calcium channel blocker, an immunosuppressive drug and a fluorescence marker.

26. The medicament preparation of claim 24, in which the compound comprising at least one thiol group has a molecular weight of at least 250 g/mol.

27. The medicament preparation of claim 24, in which the compound comprising at least one thiol group comprises at least 10 thiol groups.

28. The medicament preparation of claim 24, in which the compound comprising at least one thiol group is selected from the group consisting of a thiolated carbomer, a thiolated poly(meth)acrylic acid, a thiolated cellulose, a thiolated polyglucosamine, a thiolated polylysine, a thiolated polyarginine, a glutathione and combinations thereof.

29. The medicament preparation of claim 24, in which the compound comprising at least one thiol group comprises at least one thiol group per 500 g/mol of molecular weight.

30. The medicament preparation of claim 24, in which the compound comprising at least one thiol group is effective to reduce efflux ratio by at least 50% when being applied in a concentration of less than 100 μM.

31. The medicament preparation of claim 24, in which the preparation is configured as nanoparticles, microparticles, matrix-tablets, emulsions, solutions, suspensions, eye drops or capsules.

32. The medicament preparation of claim 24, in which said medicament preparation is a dosage form for oral, nasal, pulmonary, vaginal, buccal, rectal and ocular administration.

33. The medicament preparation of claim 24, in which the thiol containing compound is glutathione.

34. A method of increasing the uptake of a drug whose mucosal uptake is inhibited by efflux pumps, the method comprising administering the drug in a medicament preparation comprising an effective amount of a compound comprising at least one thiol group to inhibit efflux pumps.
